(19) United States
(12) Patent Application Publication
(10) Pub. No.: US 2023/0329991 A1
(43) Pub. Date: Oct. 19, 2023

(54) PERMANENT HAIR SHAPING COMPOSITION

(71) Applicant: Wella Germany GmbH, Darmstadt (DE)

(72) Inventor: Giuseppe Esposito, Frankfurt am Main (DE)

(73) Assignee: Wella Germany GmbH, Darmstadt (DE)

(21) Appl. No.: 18/044,138

(22) PCT Filed: Sep. 23, 2021
(86) PCT No.: PCT/EP2021/076237

§ 371 Date: Mar. 6, 2023

Related U.S. Application Data
(60) Provisional application No. 63/082,128, filed on Sep. 23, 2020.

(51) Int. Cl. A61K 8/46; A61K 8/34; A61K 8/86; A61K 8/41; A61Q 5/04

(52) U.S. Cl. CPC A61K 8/46; A61K 8/342; A61K 8/86; A61K 8/41; A61Q 5/04; A61K 2800/33; A61K 2800/48; A61K 2800/884

(57) ABSTRACT

A permanent shaping composition for the permanent deformation of hair, a kit, a method and a use thereof are provided. The present invention provides a superior permanent shaping performance, particularly vis-à-vis odor control, application, combing and rinsability. Particularly, the present invention provides a superior permanent shaping performance, with a reduced or an eliminated smell of the compounds with a mercapto-functional group upon application.

### TECHNICAL FIELD

The present invention relates to a permanent shaping composition for the permanent deformation of hair, a kit, a method and a use thereof. The permanent shaping composition comprises, in a cosmetically acceptable carrier, at least one reducing agent comprising a mercapto functional group, at least one alkalizing agent, at least one fatty alcohol, and at least one non-ionic surfactant, wherein the fatty alcohol and the non-ionic surfactant are present in a weight ratio from 10:3.5 to 10:1. The present invention provides a superior permanent shaping performance, particularly vis-à-vis odor control, application (e.g. pick-up and spreadability), combing and rinsability. Particularly, the present invention provides a superior permanent shaping performance, with a reduced or an eliminated smell of the compounds with a mercapto-functional group upon application.

### BACKGROUND

The classic technique for permanent hair shaping, i.e., permanent waving and straightening of hair, is familiarly on two treatment steps: In the first step, the cystine-disulfide bridges of the keratin of the hair are opened by the action of a composition that comprises a reducing ingredient (permanent shaping composition). The hair is then put into the desired shape. In a second step, cystine-disulfide bonds are closed again using a fixative, that is, a composition comprising an oxidizing ingredient.

As classic reducing agents, compounds comprising mercapto-functional groups are used, for example thioglycolic acid, ammonium or monoethanolamine salts of thioglycolic acid, 2-mercaptopropionic acid (thiolactic acid), 3-mercaptopropionic acid, cysteine, and derivatives of these compounds, cysteamine, mercaptocarboxylic acid esters, and N-alkyl-2-mercaptoacetamides.

The first step, i.e., the cystine-disulfide opening reaction, is typically carried out at a pH of 7.5 to 9 in the presence of an alkalizing agent. The alkaline-adjusted permanent shaping compositions based on mercapto-functionalized reducing agents, however, cause unpleasant odor. The unpleasant odor of the reducing agents requires intensive perfuming of the products.

Despite the fact that commercial permanent hair deformation products have been available for many years, still no products exist which effectively reduce or even completely avoid the malodor during and after the hair shaping process, and simultaneously provide an effective permanent hair shaping effect.

By using thiolactic acid, it is possible to slightly reduce the malodor. In comparison to the widely used thioglycolic acid, the permanent deformation effectiveness of thiolactic acid is, however, considerably weaker.

It is therefore an objective of the present invention to provide a composition and a method for permanent shaping of hair which significantly improve the odor during and after the shaping process of the hair, provides an effective permanent hair shaping performance, and which enhances the customer's acceptance of permanent hair shaping products.

### SUMMARY

In a first aspect, the present invention relates to a permanent shaping composition for the permanent deformation of hair comprising, in a cosmetically acceptable carrier:
(a) at least one reducing agent comprising a mercapto functional group;
(b) at least one alkalizing agent;
(c) at least one fatty alcohol;
(d) at least one non-ionic surfactant different from the fatty alcohol;
   wherein the at least one fatty alcohol and the at least one non-ionic surfactant are present in a weight ratio from about 10:3.5 to 10:1, preferably from about 10:3 to about 10:1.5, more preferably from about 10:2.5 to 10:2.0.

Expressed in molecular terms, the at least one fatty alcohol and the at least one non-ionic surfactant may be present in a molecular ratio from about 10:0.78 to 10:0.22, preferably from about 10:0.67 to about 10:0.33, more preferably from about 10:0.56 to 10:0.45.

In some embodiments, the cosmetically acceptable carrier is an aqueous carrier, preferably an aqueous carrier selected from the group consisting of water, water-soluble polyhydric alcohols and mixtures thereof.

In some embodiments, the reducing agent is selected from the group consisting of thioglycolic acid, thiolactic acid and their salts thereof, such as their ammonium and ethanolamine salts; cysteine and its salts thereof, such as hydrochloride cysteine; homocysteine; cysteamine and its salts thereof; N-acetyl cysteine; thioglycerol; ethanediol monothioglycolate; 1,2-propyleneglycol monothioglycolate; 1,3-propanediol monothioglycolate or the isomer mixture resulting therefrom; 1,3-butanediol, 1,4-butanediol monothioglycolate and their isomer mixtures therefrom; polyethylene glycol monothioglycolates, such as di-, tri- and tetraethyleneglycol monothioglycolates; glycerol monothiolactate; mercapto carboxylic acids and esters thereof; N-alkyl-2-mercaptoacetamides; and mixtures thereof; preferably from the group consisting of cysteamine, cysteine, thioglycolic acid, their salts thereof and mixtures thereof.

In some embodiments, the composition comprises a total amount of reducing agent ranging from about 0.1 to about 35%, preferably from about 0.5 to about 20%, more preferably about 1 to about 15%, of a reducing agent, by total weight of the composition.

In some embodiments, the alkalizing agent is selected from the group consisting of ammonia, ammonium carbamate, ammonium(bi)carbonate, monoethanolamine, diethanolamine, triethanolamine, 2-amino-2-methyl-1-propanol, 2-amino-2-methyl-1,3-propanediol, 2-amino-2-ethyl-1,3-propanediol, 2-amino-1-butanole, tris(hydroxylmethyl)-aminomethane, tris-(2-hydroxypropyl)-amine, lysine, 2,2-iminobisethanol, iminourea (guanidine carbonate), tetrahydro-1,4-oxazine, 2-amino-5-guanidin valeric acid, 2-aminoethansulfonic acid, N-methyl ethanolamine, isopropanolamine, diisopropanolamine, triisopropanolamine, glucamine, sodium hydroxide, potassium hydroxide, lithium hydroxide, magnesium hydroxide, other water-soluble physiologically tolerable salts of organic and inorganic bases, and mixtures thereof; preferably the alkalizing agent is selected from the group consisting of ammonia, monoethanolamine, and mixture thereof.

In some embodiments, the composition comprises a total amount of alkalizing agent ranging from about 0.1 to about 10%, preferably from about 0.2 to about 5%, more preferably from 0.25 to 4% by total weight of the composition.

In some embodiments, the fatty alcohol is selected from the group consisting of linear and/or branched C₁₄ to C₃₀ fatty alcohols; preferably from the group consisting of linear C₁₄ to C₃₀ fatty alcohols; more preferably from the group consisting of cetyl alcohol, stearyl alcohol, cetearyl alcohol, behenyl alcohol, and mixtures thereof; still more preferably the fatty alcohol is cetearyl alcohol.

In some embodiments, the composition comprises a total amount of fatty alcohol ranging from about 0.5 to about 20%, preferably from about 2 to about 15%, more preferably from about 4 to about 12%, still more preferably from about 5 to about 10% by total weight of the composition;

In some embodiments, the non-ionic surfactant is selected from the group consisting of polyoxyethylene C₈ to C₃₀ alkyl ethers; preferably from the group consisting of polyoxyethylene C₈ to C₃₀ alkyl ethers having at least 2 ethylene oxide units, or at least 5 ethylene oxide units; more preferably from the group consisting of polyoxyethylene C₈ to C₃₀ alkyl ethers having from 10 to 50 ethylene oxide units; still more preferably from the group consisting of polyoxyethylene C₈ to C₃₀ alkyl ethers having from 15 to 30 ethylene oxide units; still more preferably from the group consisting of ceteareth-25, steareth-20, and mixtures thereof; most preferably the non-ionic surfactant is steareth-20.

In some embodiments, the composition comprises a total amount of non-ionic surfactant other than fatty alcohol ranging from about 0.2 to about 5%, preferably from about 0.5 to about 4%, more preferably from about 1.1 to about 2.2%, by total weight of the composition .

In some embodiments, the composition comprises a total amount of fatty alcohol and non-ionic surfactant ranging from about 0.7 to about 25%, preferably from about 2.5 to about 19%, more preferably from about 6.1 to about 12.2%, by total weight of the composition; and wherein the fatty alcohol and the non-ionic surfactant are comprised, in part or all, in a gel network system.

In some embodiments, the composition is free of anionic surfactant and/or cationic surfactant.

In some embodiments, the composition comprises a thickener and/or rheology modifier, a liquid oil, a solid fat, a cationic polymer, a silicone, a swelling agent, a disulfide, and mixtures thereof.

In some embodiments, the composition comprises an additive selected from the group consisting of alcohols, sugar alcohols, preservatives, antioxidants, sequestering agents, salts, pearlescent aids, UV-filters and sunscreens, pH adjusting agents, opacifiers, solubilizers, perfume oils, dyes, aminoacids, hydrolysed proteins, and mixtures thereof.

In a second aspect, the present invention relates to a kit for the permanent deformation of hair comprising:
(a) an individually packaged permanent shaping composition according to any preceding claims;
(b) an individually packaged fixing composition comprising an oxidizing agent.

In a third embodiment, the present invention relates to a method for the permanent deformation of hair comprising the following steps:
(a) providing a permanent shaping composition according to any preceding claims;
(b) applying the permanent shaping composition to the hair before or after putting the hair in a desired shape;
(c) allowing the permanent shaping composition to act on the hair for a predetermined acting time sufficient for the permanent shaping of the hair;
(d) rinsing the hair with water;
(e) applying to the hair a fixing composition comprising an oxidizing agent for performing an oxidative post-treatment of the hair; and
(f) rinsing the hair with water.

### DETAILED DESCRIPTION

As used herein the term “hair” may be “living”, i.e., on a living body, or may be “non-living”, i.e., in a wig, hairpiece or other aggregation of non-living keratinous fibers. Mammalian, preferably human hair is preferred. However wool, fur and other keratin containing fibers are suitable substrates for the compositions according to the present invention.

By “permanent shaping composition” is meant a composition used in the first step of the permanent deformation of hair, i.e., for the cystine-disulfide opening reaction. The permanent shaping composition is referred hereinafter as “the composition”, unless otherwise specified.

Permanent shaping compositions according to the invention may be used for both, permanent waving (i.e. curling of human hair) and straightening, (i.e. smoothing of human hair).

By “at least one compound” or “a compound” is meant one compound or a mixture of two or more compounds belonging to the defined class of said compounds.

By “fatty alcohol” is meant a non-alkoxylated, saturated or unsaturated, linear or branched, alcohol having from 14 to 30 carbon atoms.

All percentages are by weight of the permanent shaping composition, i.e. of the ready-to-use composition which is the composition to be applied on hair, unless otherwise specified. Also ratios are weight ratios unless specifically stated otherwise.

### Permanent Shaping Composition

The first aspect of the present invention is a permanent shaping composition for the permanent deformation of hair comprising, in a cosmetically acceptable carrier:
(a) at least one reducing agent comprising a mercapto functional group;
(b) at least one alkalizing agent;
(c) at least one fatty alcohol;
(d) at least one non-ionic surfactant different from the fatty alcohol;
   wherein the at least one fatty alcohol and the at least one non-ionic surfactant are present in a weight ratio from about 10:3.5 to about 10:1, preferably from about 10:3 to about 10:1.5, more preferably from about 10:2.5 to 10:2.0.

### Cosmetically Acceptable Carrier

The permanent shaping composition comprises a cosmetically acceptable carrier or solvent.

The solvent may be an aqueous carrier, preferably an aqueous carrier selected from the group consisting of water, water-soluble polyhydric alcohols and mixtures thereof.

Water-soluble polyhydric alcohols may have two or more hydroxyl groups in the molecule.

Polyhydric alcohols may be selected from the group consisting of dihydric alcohols such as ethylene glycol, 1,2- or 1,3-propanediol, 1,2-pentanediol and glycerin; further, trimethylene glycol, 1,2-butylene glycol, 1,3-butylene glycol, 1,4-butylene glycol, tetramethylene glycol, 2,3-butylene glycol, pentamethylene glycol, 2-butene-1,4-diol, hexylene glycol, octylene glycol; trihydric alcohols such as glycerin, trimethylol propane, 1,2,6-hexanetriol and the like; tetrahydric alcohols such as penthaerythritol; pentahydric alcohols such as xylytol, etc.; hexahydric alcohols such as sorbitol, mannitol; polyhydric alcohol polymers such as diethylene glycol, dipropylene glycol, polyethylene glycol, polypropylene glycol, tetraethylene glycol, diglycerine, polyethylene glycol, triglycerine, tetraglycerine, polyglycerine; dihydric alcohol alkyl ethers such as ethylene glycol monomethyl ether, ethylene glycol monoethyl ether, ethylene glycol monobutyl ether, ethylene glycol monophenyl ether, ethylene glycol monohexyl ether, ethylene glycol mono-2-methylhexyl ether, ethylene glycol isoamyl ether, ethylene glycol benzyl ether, ethylene glycol isoprapyl ether, ethylene glycol dimethyl ether, ethylene glycol diethyl ether, ethylene glycol dibutyl ether; dihydric alcohol alkyl ethers such as diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methyl ethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether; dihydric alcohol ether esters such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate; and mixtures thereof.

In a particularly preferred embodiment, the water-soluble polyhydric alcohol is propylene glycol, glycerin and mixture thereof.

The composition may comprise water as a main ingredient, particularly the composition may comprise a total amount of water ranging from at least about 50%, preferably from at least about 55%, more preferably from at least about 60%, by total weight of the composition. The composition may comprise a total amount of water from about 50 to about 90%, preferably from about 55 to about 85%, more preferably from about 60 to about 80%, by total weight of the composition.

The composition may comprise a total amount of water-soluble polyhydric alcohols ranging from about 0.1 to about 10%, preferably from about 0.5 to about 8%, more preferably from about 2 to 6%, by total weight of the composition.

### Reducing Agent

The permanent shaping composition comprises at least one reducing agent. The reducing agent comprises a mercapto-functional group. Generally, all reducing agents known in the art, which are suitable for use in the permanent deformation of hair, can be used.

The reducing agent may be selected from the group consisting of thioglycolic acid, thiolactic acid and their salts thereof, such as their ammonium and ethanolamine salts; cysteine and its salts thereof, such as hydrochloride cysteine; homocysteine; cysteamine and its salts thereof; N-acetyl cysteine; thioglycerol; ethanediol monothioglycolate; 1,2-propyleneglycol monothioglycolate; 1,3-propanediol monothioglycolate or the isomer mixture resulting therefrom; 1,3-butanediol, 1,4-butanediol monothioglycolate and their isomer mixtures therefrom; polyethylene glycol monothioglycolates, such as di-, tri- and tetraethyleneglycol monothioglycolates; glycerol monothiolactate; mercapto carboxylic acids and esters thereof; N-alkyl-2-mercaptoacetamides; and mixtures thereof.

The use of inorganic reducing sulfur compounds such as sodium hydrogen sulfite is basically also possible.

Preferably, the reducing agent is selected from the group consisting of cysteamine, cysteine, thioglycolic acid, their salts thereof and mixtures thereof.

The permanent shaping composition may comprise a total amount of reducing agent ranging from at least about 0.1%, preferably from at least about 0.5%, more preferably from at least about 1%, by total weight of the composition. Particularly, the composition may comprise a total amount of reducing agent ranging from about 0.1 to about 35%, preferably from about 0.5 to about 20%, more preferably about 1 to about 15%, of a reducing agent, by total weight of the composition.

### Alkalizing Agent

The permanent shaping composition comprises at least one alkalizing agent.

The alkalizing agent may be selected from the group consisting of ammonia, ammonium carbamate, ammonium(bi)carbonate, monoethanolamine, diethanolamine, triethanolamine, 2-amino-2-methyl-1-propanol, 2-amino-2-methyl-1,3-propanediol, 2-amino-2-ethyl-1,3-propanediol, 2-amino-1-butanole, tris(hydroxylmethyl)-aminomethane, tris-(2-hydroxypropyl)-amine, lysine, 2,2-iminobisethanol, iminourea (guanidine carbonate), tetrahydro-1,4-oxazine, 2-amino-5-guanidin valeric acid, 2-aminoethansulfonic acid, N-methyl ethanolamine, isopropanolamine, diisopropanolamine, triisopropanolamine, glucamine, sodium hydroxide, potassium hydroxide, lithium hydroxide, magnesium hydroxide, other water-soluble physiologically tolerable salts of organic and inorganic bases, and mixtures thereof; preferably the alkalizing agent is selected from the group consisting of ammonia, monoethanolamine, and mixture thereof.

By varying the pH value, a permanent shaping composition can be made available that is universally suitable for every hair structure, optionally with the additional application of heat.

The composition brings about an elastic, durable and uniform waving from the root of the hair to the ends, without eliciting allergic or sensitizing reactions. It is desirable to adjust the pH value to between about 5.5 and 12, preferably about 6.5 and 10.5, most preferably about 7.5 to 9.5. Thus, the quantity of the alkalizing agent is dependent on the reducing agent and the desired pH value of the composition.

The permanent shaping composition may comprise a total amount of alkalizing agent ranging from about 0.1 to about 10%, preferably from about 0.2 to about 5%, more preferably from 0.25 to 4% by total weight of the composition.

### Buffer

The composition may comprise a buffer salt. According to the present invention, the buffer may be at least selected from the group consisting of bicarbonate salts, preferably the buffer salt is ammonium bicarbonate.

### Fatty Alcohols

The composition comprises at least one fatty alcohol. The fatty alcohol may be selected from the group consisting of linear and/or branched C₁₄ to C₃₀ fatty alcohols; preferably from the group consisting of linear C₁₄ to C₃₀ fatty alcohols; more preferably from the group consisting of cetyl alcohol, stearyl alcohol, cetearyl alcohol, behenyl alcohol, and mixtures thereof. In a particularly preferred embodiment, the fatty alcohol is cetearyl alcohol.

The composition may comprise a total amount of fatty alcohol ranging from about 0.5 to about 20%, preferably from about 2 to about 15%, more preferably from about 4 to about 12%, still more preferably from about 5 to about 10% by total weight of the composition. The amount of the fatty alcohol described hereinbefore can account for up to 100% (or 100%) of the total amount of fatty alcohol in the composition.

### Non-Ionic Surfactants

The composition comprises at least one non-ionic surfactant other than the fatty alcohol. The non-ionic surfactant may be selected from non-ionic surfactant comprising one or more polyethyleneoxide chain including the following compounds: polyoxyethylene alkyl ethers, polyethyleneglycol fatty acid esters, polyoxyethylene castor oil, polyoxyethylene hydrogenated castor oil, polyoxyethylene fatty amides and their monoethanolamine and diethanolamine derivatives and polyethoxylated fatty amines, and mixtures thereof. The non-ionic surfactant(s) may be selected from the group consisting of polyoxyethylene C₈ to C₃₀ alkyl ethers; preferably from the group consisting of polyoxyethylene C₈ to C₃₀ alkyl ethers having at least 2 ethylene oxide units, or at least 5 ethylene oxide units; more preferably from the group consisting of polyoxyethylene C₈ to C₃₀ alkyl ethers having from 10 to 50 ethylene oxide units; still more preferably from the group consisting of polyoxyethylene C₈ to C₃₀ alkyl ethers having from 15 to 30 ethylene oxide units; most preferably from the group consisting of ceteareth-25, steareth-20, and mixtures thereof. In a particularly preferred embodiment, the non-ionic surfactant is steareth-20.

The composition may comprise a total amount of non-ionic surfactant other than the fatty alcohol ranging from about 0.2 to about 5%, preferably from about 0.5 to about 4%, more preferably from about 1.1 to about 2.2%, by total weight of the composition. The amount of the non-ionic surfactant (or the mixture of non-ionic surfactants thereof) described hereinbefore can account for up to 100% (or 100%) of the total amount of non-ionic surfactant in the composition.

### Gel Network System

The composition may comprise a total amount of fatty alcohol and non-ionic surfactant as described hereinbefore ranging from about 0.7 to about 25%, preferably from about 2.5 to about 19%, more preferably from about 6.1 to about 12.2%, by total weight of the composition. The fatty alcohol(s) and the non-ionic surfactant(s) may be comprised in the composition, in part or all, in a gel network system.

The formation of the gel network involves heating a dispersion of the fatty alcohol in water with the surfactant to a temperature above 80° C. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature the liquid crystal phase is converted into a solid crystalline gel network. The gel network may particularly contribute in reducing, or even eliminating, the odour of the compounds comprising a mercapto-functional group.

From about 50 to about 100%, preferably from about 70 to about 100%, more preferably from about 90 to about 100%, most preferably about 100% of the fatty alcohol present in the composition may be comprised within the gel network. From about 50 to about 100%, preferably from about 70 to about 100%, more preferably from about 90 to about 100%, most preferably about 100% of the non-ionic surfactant present in the composition may be comprised within the gel network.

The inventors have surprisingly found that the odour of compounds comprising a mercapto-functional group, which is particularly noticeable during application and leave of the permanent shaping composition onto hair, can be significantly reduced, or even eliminated, by carefully selecting and using in combination specific classes of compounds. Indeed, the inventors have found that the reduction or elimination of smell is achieved with fatty alcohols and non-ionic surfactants, wherein the fatty alcohols and the non-ionic surfactants are comprised in the composition at a specific ratio as defined hereinbefore.

### Surfactants Other than Non-Ionic Surfactants

The composition may further comprise at least one surfactant, other than the non-ionic surfactant. Suitable surfactant(s) generally have a lipophilic chain length of from 8 to 30 carbon atoms and may be selected from anionic surfactants, amphoteric surfactants, cationic surfactants, and mixtures thereof. Any suitable surfactant known in the art may be used.

The composition comprises a total amount of anionic surfactant of less than about 0.5%, preferably less than about 0.3%, more preferably less than about 0.1%, by total weight of the composition. Alternatively, the composition may be free of anionic surfactant.

The composition comprises a total amount of cationic surfactant of less than about 0.5%, preferably less than about 0.3%, more preferably less than about 0.1%, by total weight of the composition. Alternatively, the composition may be free of cationic surfactant.

The inventors have surprisingly found that a greater reduction of odour of the compounds comprising a mercapto-functional group is achieved when the permanent shaping composition is free of anionic and/or cationic surfactant(s). Indeed, it is believed that incorporating anionic and/or cationic surfactant(s) into the composition may alter the gel network formation, the presence of said gel network being necessary for achieving the odour reduction of the compounds comprising a mercapto-functional group upon application onto hair.

### Thickeners and/or Rheology Modifiers

The composition may further comprise at least one thickener and/or at least one rheology modifier in an amount sufficient to provide the composition with a viscosity so that it can be readily applied to the hair without unduly dripping off the hair and causing mess.

Suitable thickeners include, but are not limited to: associative polymers, polysaccharides, non-associative polycarboxylic polymers, and mixtures thereof. Commercially available salt-tolerant thickeners include, but not limited to: xanthan gum, diutan gum, tara gum, guar, hydroxypropyl guar, scleroglucan, methylcellulose, ethyl cellulose (commercially available as Aquacote), hydroxyethyl cellulose (Natrosol), carboxymethyl cellulose, hydroxypropylmethyl cellulose, microcrystalline cellulose, hydroxybutylmethyl cellulose, hydroxypropyl cellulose (Klucel), hydroxyethyl ethyl cellulose, cetyl hydroxyethyl cellulose (Natrosol Plus 330), N-vinylpyrollidone (Povidone), Acrylates/Ceteth-20 Itaconate Copolymer (Structure 3001), hydroxypropyl starch phosphate (Structure ZEA), polyethoxylated urethanes or polycarbamyl polyglycol ester such as PEG-150/Decyl/SMDI copolymer (Aculyn 44), PEG-150/Stearyl/SMDI copolymer (Aculyn 46), trihydroxystearin (Thixcin), acrylates copolymer (Aculyn 33) or hydrophobically modified acrylate copolymers (such as Acrylates/Steareth-20 Methacrylate Copolymer as Aculyn 22), acrylates/steareth-20 methacrylate crosspolymer (Aculyn 88), acrylates/vinyl neodecanoate crosspolymer (Aculyn 38), acrylates/beheneth-25 methacrylate copolymer (Aculyn 28), acrylates/Clü-30 alkyl acrylate crosspolymer (Carbopol ETD 2020), non-ionic amphophilic polymers comprising at least one fatty chain and at least one hydrophilic unit selected from polyether urethanes comprising at least one fatty chain, and mixtures thereof. In on preferred embodiment, the thickener is xantham gum.

The composition may comprise a total amount of thickener ranging from at least about 0.1%, preferably at least about 0.2%, preferably at least about 0.25%, more preferably from 0.25 to 2%, by total weight of the composition.

### Viscosity

According to the present invention the compositions have a viscosity of from 100 to 2000 mPas, preferably from 200 to 1500 mPas, more preferably from 500 to 1000 mPas. Viscosity is determined according to the test method defined hereinafter.

### Viscosity Test Method

Viscosity measurements are carried out on a controlled stress rheometer of AR500, AR1000 or AR2000 type manufactured by TA Instruments, or equivalent instrument. A 6 cm flat acrylic cross hatched parallel plate geometry (TA item 518600.901) and a stainless-steel cross hatched base plate (TA item 570011.001) are used. The rheometer is prepared for flow measurements as per standard manufacturer procedure. The parallel plate geometry gap is set to 1000 microns.

The flow procedure is programmed to the rheometer with the following conditions: continuous stress ramp 0.1-300 Pa over 2 minutes at 25° C., including 250 measurement points in linear mode. The product is loaded into the geometry as per standard procedure. Shear stress value at 10 sec⁻¹ shear rate is obtained from the shear stress vs. shear rate curve, and the corresponding viscosity is calculated by dividing the obtained shear stress by 10.

### Liquid Oils

The permanent shaping composition may comprise a liquid oil. The liquid oil may be selected from the group consisting of avocado oil, tsubaki oil, turtle oil, macadamia nuts oil, corn oil, mink oil, olive oil, rape seed oil, yolk oil, sesame oil, parsic oil, wheat germ oil, sasanqua oil, castor oil, linseed oil, safflower oil, cotton seed oil, perilla oil, soybean oil, peanut oil, tea seed oil, kaya oil, rice bran oil, Chinese tung oil, Japanese tung oil, jojoba oil, germ oil, triglycerine, trioctanoic acid glycerine, triisopalmitic acid glycerine, liquid lanolin, hexyl laurate, liquid hydrocarbon esters such as fluid paraffin, mineral oil, isododecane, fatty acid oils, squalene, ester oils such as cetyl octanoate, isopropyl myristate and mixtures thereof; preferably the liquid oil is mineral oil.

The composition may comprise a total amount of liquid oil ranging from at least about 0% to at least about 5%, by total weight of the composition.

### Solid Fats and Waxes

The permanent shaping composition may comprise a solid fat. The solid fat may be selected from the group consisting of cacao fat, coconut oil, horse fat, hardened coconut fat, palm oil, tallow, sheep fat, hardened tallow, palm kernel oil, lard, ox bone fat, wood wax kernel oil, hardened castor oil; lanolin, kapok wax, lanolin acetate, isopropyl lanolin fatty acid, reduced lanolin, hard lanolin, POE lanolin alcohol ether, POE lanolin alcohol acetate, POE cholesterol ether, lanolin fatty acid polyethylene glycol, POE hydrogenated lanolin alcohol ether; hydrocarbons, non-volatile hydrocarbons and solid hydrocarbon esters such as solid paraffin, vaseline, ozocerite, pristan, ceresin, petrolatum, microcrystalline wax; phospholipides, e.g. lecithines or ceramides, beeswax, apple wax, canderilla wax, cotton wax, carunauba wax, bayberry wax, insect wax, whale wax, montan wax, rice bran wax, kapok wax, cane wax, jojoba wax, and shellac wax; preferably the solid fat is petrolatum. The wax is not a fatty alcohol. The composition may comprise a total amount of solid fat ranging from at least about 0% to at least about 5%, by total weight of the composition.

### Cationic Polymers

The permanent shaping composition may comprise at least one cationic polymer. Cationic polymers may be selected from the group consisting of cationic homopolymers, cationic copolymers, cationic polysaccharides, cationic silicon polymers comprising quaternary nitrogen groups such as for example quaternium-80, and mixtures thereof.

The composition may comprise a total amount of cationic polymer ranging from about 0.05 to about 15%, preferably from about 0.1 to about 8%, more preferably from about 0.2 to about 3%, by total weight of the composition.

Suitable cationic homopolymers are made by homopolymerizing cationic monomers comprising quaternary nitrogen groups while suitable cationic copolymers are made by copolymerizing cationic monomers comprising quaternary nitrogen groups and non-cationic monomers. Suitable cationic monomers are unsaturated, radical polymerizable compounds carrying at least one cationic group, particularly ammonium-substituted vinyl monomers such as trialkyl methacryl oxyalkyl ammonium, trialkyl acryloxyalkyl ammonium, dialkyl diallyl ammonium, and quaternary vinyl ammonium monomers with groups containing cyclic, cationic nitrogen such as pyridinium, imidazolium, or quaternary, e.g. alkyl vinyl imidazolium, alkyl vinyl pyridinium, or alkyl vinyl pyrrolidone salts.

The alkyl groups of these monomers are preferably lower alkyl groups, such as C₁ to C₇ alkyl groups, with C₁ to C₃ alkyl groups being especially preferred. The ammonium group-containing monomers can be copolymerized with non-cationic monomers. Suitable comonomers are, for example, acrylamide, methacrylamide, alkyl- and dialkyl acrylamide, alkyl and dialkyl methacrylamide, alkyl acrylate, alkyl methacrylate, vinyl caprolactone, vinyl caprolactam, vinyl pyrrolidone, vinyl ester, e.g. vinyl acetate, vinyl alcohol, propylene glycol, or ethylene glycol, wherein the alkyl groups of these monomers are preferably C₁ to C₇ alkyl groups, with C₁ to C₃ alkyl groups being especially preferred.

Suitable cationic polysaccharides comprising quaternary nitrogen groups are for example cationic derivatives of cellulose, starch or guar, chitosan and chitosan derivatives.

Preferred cationic homopolymer is polyquaternium-6 and preferred cationic copolymer is polyquaternium-11

### Silicones

Silicones may be volatile or non-volatile, and soluble or insoluble; preferably insoluble silicones. By “soluble” is meant that the silicone is miscible with the aqueous carrier of the composition so as to form part of the same phase. By “insoluble” is meant that the silicone forms a separate, discontinuous phase from the aqueous carrier, such as in the form of an emulsion or a suspension of droplets of the silicone.

Soluble silicones may be selected from the group consisting of silicone copolyols, such as dimethicone copolyols, e.g. polyether siloxane-modified polymers, such as polypropylene oxide, polyethylene oxide modified polydimethylsiloxane, wherein the level of ethylene and/or propylene oxide sufficient to allow solubility in the composition.

The insoluble silicones may have a viscosity of from about 1,000 to about 2,000,000 mPas at 25° C., preferably from about 10,000 to about 1,800,000 mPas, more preferably from about 100,000 to about 1,500,000 mPas. The viscosity can be measured by means of a glass capillary viscometer as set forth in Dow Corning Gorparate Test Method CTM0004, Jul. 20, 1970.

The insoluble silicone may be selected from the group consisting of polyalkyl siloxanes, polyaryl siloxanes, polyalkylaryl siloxanes, polyether siloxane copolymers, dimethylpolysiloxane containing terminal hydroxyl groups, methylphenyl polysiloxane containing terminal hydroxyl groups and mixtures thereof, low molecular weight oligomeric polydimethylsiloxane or cyclic polydimethylsiloxane, polyether siloxane copolymers, Silicone conditioning polymers that are specially preferred are CTFA: dimethicone, amodimethicone, dimethicone bisamino hydroxypropyl copolyol, bisamino PEG/PPG-41/3 aminoethyl PG-propyl dimethicone, dihydroxypolydimethylsiloxane (CTFA: dimethiconol). Also preferred are volatile silicones such as e.g. CTFA: dimethicone, dimethicone copolyol and cyclodimethicone; preferably the insoluble silicone may be selected from the group consisting of dimethicone, amodimethicone, or mixtures thereof.

The composition may comprise a total amount of silicone ranging from about 0 to about 6%, by total weight of the composition.

### Swelling Agents

So-called swelling agents and penetrating substances may be added to the hair shaping composition, examples being urea, melamine, ethers, e. g. dipropylene glycol monomethyl ether, 2-pyrrolidon, imidazolidin-2-one, 1-methyl-2-pyrrolidone; alkali or ammonium thiocyanate, polyvalent alcohols, isopropanol; preferably the swelling agent and penetrating substance is urea.

The composition may comprise a total amount of swelling agents ranging from about 0% to about 5%, by total weight of the composition.

### Disulfides

It is advantageous if the permanent shaping agent, to avoid making the hair too kinky, contains the disulfide of a hair keratin-reducing compound (thiol), particularly dithioglycolic acid, 2,2′dithio-bis[N-(3-hydroxypropyl)-acetamid], 2,2′-dithio-bis[N-(propyl)-acetamid], 2,2′dithio-bis[N-(2-hydroxypropyl)-acetamid], dithiolactic acid and their salts.

The composition may comprise a total amount of disulfide ranging from about 0.1 to about 30%, more preferably from about 0.5 to about 20%, and most preferred from about 1 to about 10%, based on the total weight of the composition. The ratio between the hair keratin-reducing agents and the disulfides is preferably from about 20:1 to about 1:2, and more preferably from 10:1 to 1:1.

In a preferred embodiment, the disulphide is selected from the group consisting of cystamine, cystine and dithiodiglycolic acid, their salts thereof, and mixtures thereof.

### Additives

The composition may further comprise usual and known additives for permanent hair shaping compositions. Suitable additives are for example: alcohols such as ethanol, propanol, isopropanol; sugar alcohols such as sorbitol, maltitol, maltotriose, mannitol, sucrase, erythritol, glucose, fructose, starch sugar, maltose, xylytose, starch sugar reduced alcohol; preservatives such as benzyl alcohol, methyl paraben, propyl paraben and imidazolidinyl urea; antioxidants such as tocopheroles; sequestering agents such as disodium ethylenediamine tetraacetate, ethylenediamine-N,N′-disuccinic acid and its salts; salts such as sodium chloride, potassium chloride, ammonium chloride, sodium sulfate, potassium sulfate and ammonium sulfate salts such as sodium chloride, potassium chloride, ammonium chloride, sodium sulfate, potassium sulfate and ammonium sulfate; pearlescent aids such as ethylene glycol distearate and Mica; UV-filters and sunscreens, e.g. such as p-methoxy cinnamic acid isoamylester, lipophile cinnamic acid esters, salicylic acid esters, 4-amino benzoic acid derivatives or hydrophilic sulfonic acid derivatives of benzophenones or 3-benzyliden campher; pH adjusting agents, such as citric acid, formic acid, glyoxylic acid, acetic acid, lactic acid, pyruvic acid, sodium citrate, succinic acid, phosphoric acid, sodium hydroxide, sodium carbonate; opacifiers such as kaolin and titanium oxide; solubilizers; perfume oils; dyes; aminoacids and/or hydrolysed proteins; and mixtures thereof.

The additives are preferably used in quantities usual for such purposes; from about 0.1 to about 20% of alcohols; from about 0.1 to about 5% of each of the sugar alcohols, salts, pearlescent aids and UV-filters and sunscreens; from about 0.01 to about 1% of each of antioxidants, pH adjusting agents, sequestering agents, opacifiers, preservatives, perfume oils, solubilizers, aminoacids and hydrolysed proteins and/or dyes, by total weight of the composition.

### Kit for the Permanent Deformation of Hair

In a second aspect, the present invention relates to a kit for the permanent deformation of hair comprising:
(a) an individually packaged permanent shaping composition comprising at least one reducing agent comprising a mercapto functional group, at least one alkalizing agent, at least fatty alcohol, at least one non-ionic surfactant different from the fatty alcohol; wherein the at least one fatty alcohol and the at least one non-ionic surfactant are present in a weight ratio from about 10:3.5 to 10:1, preferably from about 10:3 to about 10:1.5, more preferably from about 10:2.5 to 10:2.0;
(b) an individually packaged fixing composition comprising an oxidizing agent.

“Individually packaged” means that the components may be packaged in separate containers or in compartmented containers without being in contact with each other.

In the kit for the permanent deformation of hair, the carrier, the reducing agent, the alkalizing agent, the fatty alcohol, the non-ionic surfactant, and any optional compounds are in accordance with the compounds described with regard to the first aspect of the invention.

The fixing composition is a composition for oxidatively post-treating (fixing) the hair comprising an oxidizing agent.

Any known oxidizing agents that have been used before in fixing compositions can be used for the fixation. Examples of such oxidizing agents are potassium bromate, sodium bromate, sodium perborate, dehydroascorbic acid, urea peroxide, hydrogen peroxide, and inorganic peroxygen materials capable of yielding hydrogen peroxide in an aqueous solution. Such materials are for example inorganic alkali metal peroxides (such as sodium periodate and sodium peroxide); organic peroxides (such as urea peroxide and carbamide peroxide); inorganic perhydrate salt bleaching compounds (such as the alkali metal salts of perborates, percarbonates, perphosphates, persilicates, persulphates and the like); and mixtures thereof. Inorganic perhydrate salts may be incorporated for example as monohydrates, or tetrahydrates. Alkyl/aryl peroxides and/or peroxidases may also be used. Mixtures of two or more such oxidizing agents can be used if desired. The oxidizing agents may be provided in aqueous solution or as a powder which is dissolved prior to use. Hydrogen peroxide is preferred.

The concentration of the oxidizing agent varies, depending on the application time and the application temperature. Generally, oxidizing agents are used in a concentration of from about 0.5 to 12%, preferably from about 1 to about 3%, by total weight of the fixing composition.

The fixing composition can comprise other materials, for example, weak acids or peroxide stabilizers. The fixing composition may also include other substances, such as wetting agents, hair-care substances such as cationic polymers, weak acids, buffer substances or peroxide stabilizers, and may be in the form of an aqueous solution, an emulsion, or a thickened water-based form, in particular a cream, gel or paste. These typical additives may be contained in the fixing composition in a quantity of from about 0.1 to about 10%, by total weight of the fixing composition.

The fixing composition can be present in the form of an aqueous solution or an emulsion, as well as in a thickened form on an aqueous basis, particularly as a cream, gel, or paste. It is also possible to fill these compositions into aerosol cans under pressure and to release them as aerosol foam. Preferably, the fixing composition is an aqueous solution or emulsion.

The permanent shaping and fixing compositions may be manufactured by conventional processes known in the art for manufacturing permanent shaping and fixing products, e.g., by admixing the ingredients of each composition in suitable vessels, followed by packaging in appropriate individual containers.

### Method for the Permanent Deformation of Hair

In a third aspect, the present invention relates to a method for the permanent deformation of hair comprising the following steps:
(a) providing a permanent shaping composition comprising at least one reducing agent, at least one alkalizing agent, at least one fatty alcohol and at least non-ionic surfactant different from the fatty alcohol; wherein the at least one fatty alcohol and the at least one non-ionic surfactant are present in a weight ratio from about 10:3.5 to 10:1, preferably from about 10:3 to about 10:1.5, more preferably from about 10:2.5 to 10:2.0;
(b) applying the permanent shaping composition to the hair before or after putting the hair in a desired shape;
(c) allowing the permanent shaping composition to act on the hair for a predetermined acting time sufficient for the permanent shaping of the hair;
(d) rinsing the hair with water;
(e) applying to the hair a fixing composition comprising an oxidizing agent for performing an oxidative post-treatment of the hair; and (
(f) rinsing the hair with water.

In the method for the permanent deformation of hair according to the present invention, the carrier, the reducing agent, the alkalizing agent, the fatty alcohols, the non-ionic surfactants and any optional compounds are in accordance with the compounds described with regard to the first aspect of the invention.

The method comprises as a first step the provision of a permanent shaping composition.

Either before or after applying the permanent shaping composition, the hair is put under mechanical tension into the desired shape preferably as follows: First the hair (which is washed and towel-dried) is separated into multiple sections and then these sections are rolled onto curlers. The curlers used for permanent waves have a diameter of about 5 to 13 mm. The curlers may be connected to a digital perm machine for heating the curler and the hair. After the hair is rolled on curlers, the curlers are thoroughly wetted down using the permanent shaping composition. As an alternative, the hair may be thoroughly wetted down using the permanent shaping composition before being curled on the rollers, while the procedure of rolling the hair onto curlers is the same.

After applying the permanent shaping composition, hair may be covered with a moisture barrier.

The permanent shaping composition is then allowed to act on the hair for a predetermined acting time sufficient for the permanent shaping of the hair. The amount of time the permanent shaping composition stays on the hair is from about 2 to 45 min, preferably from about 5 to about 30 min, preferably from about 5 to about 20 min. This time can be shortened by adding heat via the use of a heat radiator or a hood dryer. This time is dependent from the hair quality, the pH value, the desired level of change as well as on the application temperature.

For example, during the predetermined acting time, hair may be heated, preferably from 50 to 150° C., more preferably from 60 to 140° C.; still more preferably from 90 to 120° C. The heating step may be carried out using a separate heating device e.g. heating hood or using the curlers themselves, by connecting them to a digital perm machine.

After the acting time, the tension is released from hair, and the moisture barrier is removed, if present.

The hair is then rinsed with water.

As a further step, the method comprises the application of a fixing composition comprising an oxidizing agent for performing an oxidative post-treatment of the hair. Depending on the hair thickness and length, the amount of fixing composition, the application time and the application temperature can vary. Preferred application times are in the range of from about 1 to about 40 min, for example from about 2 to about 20 min, more preferably from about 3 to about 10 min.

Preferred application temperatures are in the range of from about 25 to about 40° C.

After the oxidative post-treatment, the hair is rinsed with water. The curlers may be removed from the hair at any time after the oxidative post-treatment, preferably before the hair is rinsed with water.

While the method described above has been directed to permanently waving hair, the method may generally also be applied to straighten hair. Generally, hair that has been permanently waved or that is naturally curly may be straightened using the methods of the present invention.

To straighten hair the composition is applied to dry hair that is then combed, or physically smoothed or otherwise tensioned for up to about 20 minutes to allow sufficient bond breaking to occur, followed by neutralization.

### Use

The present invention also relates to the use of the composition described above, for the permanent deformation of hair.

### EXAMPLES

### Preparation of the Permanent Shaping/Straightening Compositions

The formation of the gel network involves heating a dispersion of the fatty alcohol in water with the surfactant to a temperature above 80° C. During the mixing process, the fatty alcohol melts, allowing the surfactant to partition into the fatty alcohol droplets. The surfactant brings water along with it into the fatty alcohol. This changes the isotropic fatty alcohol drops into liquid crystalline phase drops. When the mixture is cooled below the chain melt temperature the liquid crystal phase is converted into a solid crystalline gel network. The gel network may particularly contribute in reducing, or even eliminating, the odour of the compounds comprising a mercapto-functional group. Then, the other materials are added to the mixture obtained.

### Permanent Shaping Composition

| Materials | C1 | | |
|---|---|---|---|
| (weight percent) | (reference) | C2 | C4 |
| Cetearyl alcohol | 6.00 | 5.74 | 5.74 |
| Steareth-20 | — | 1.26 | 0.52 |
| Ceteareth-25 | 2.50 | — | — |
| Propylene glycol | — | 4.00 | 4.00 |
| Ammonium thioglycolate | 20.54 | 21.38 | 21.38 |
| (59.3% in water) | | | |
| Diammonium dithiodiglycolate | 4.00 | 4.00 | 4.00 |
| (48% in water) | | | |
| Ammonia | 4.00 | 2.00 | 2.00 |
| (25% in water) | | | |
| Ammonium bicarbonate | 3.70 | 1.00 | 1.00 |
| Isopropyl myristate | 2.00 | — | — |
| Beeswax | 1.00 | — | — |
| Perfume | 0.80 | 0.80 | 0.80 |
| Additional care ingredients and | 5.50 | 5.50 | 5.50 |
| additives | | | |
| water | Q.s. 100% | Q.s. 100% | Q.s. 100% |
| Ratio fatty alcohols/non-ionic | 2.40 | 4.60 | 11.04 |
| surfactants | (10:4.17) | (10:2.17) | (10:0.9) |

### Permanent Shaping Method

The permanent shaping method is conducted as follows:
(a) wash the hair with a shampoo then tower-dry the hair;
(b) apply the permanent shaping composition to the hair before putting the hair in a desired shape;
(c) allow the permanent shaping composition to act on the hair for a predetermined acting time;
(d) rinse the hair with water;
(e) blow-dry the hair until it is 90% dry;
(f) hair is separated into multiple sections and then these sections are rolled onto curlers;
(g) the curlers are connected to a digital perm machine for heating the curler and the hair;
(h) apply to the hair a fixing composition comprising an oxidizing agent for performing an oxidative post-treatment of the hair; and
(i) remove the curlers and rinse the hair with water.

### Results

| Criteria | C2 (versus C1) |
|---|---|
| Mass appearance and odour | Less odour, pleasant |
| Application | Easier to spread |
| Rinsability | Easier to rinse |
| Wet feel | Smooth slippery feel event at ends |
| Wet curl formation | More bounce and elasticity |
| Dry feel | Soft and smooth feel, less frizz |
| Dry curl formation | More bounce and elasticity |

### Permanent Straightening Composition

| Materials | C1 | | |
|---|---|---|---|
| (weight percent) | (reference) | C3 | C5 |
| Cetearyl alcohol | 6.00 | 6.75 | 6.75 |
| Steareth-20 | — | 1.48 | 0.61 |
| Ceteareth-25 | 2.50 | — | — |
| Propylene glycol | — | 4.00 | 4.00 |
| Sodium sulfate | 1.00 | 1.00 | 1.00 |
| Ammonium thioglycolate | 15.37 | 21.38 | 21.38 |
| (59.3% in water) | | | |
| Diammonium dithiodiglycolate | 8.00 | 4.00 | 4.00 |
| (48% in water) | | | |
| Ammonia | 3.30 | 2.00 | 2.00 |
| (25% in water) | | | |
| Ammonium bicarbonate | 1.0 | 1.00 | 1.00 |
| Isopropyl myristate | 2.00 | — | — |
| Beeswax | 1.00 | — | — |
| Perfume | 0.80 | 0.80 | 0.80 |
| Additional care ingredients and | 5.50 | 5.50 | 5.50 |
| additives | | | |
| water | Q.s. 100% | Q.s. 100% | Q.s. 100% |
| Ratio fatty alcohols/non-ionic | 2.40 | 4.60 | 11.06 |
| surfactants | (10:4.17) | (10:2.17) | (10:0.9) |

### Permanent Straightening Method

(a) optionally wash the hair with a shampoo then tower-dry the hair;
(b) apply the permanent shaping composition to the hair;
(c) allow the permanent shaping composition to act on the hair for a predetermined acting time;
(d) rinse the hair with water;
(e) blow-dry the hair;
(f) hair is separated into multiple sections and then straighten with straightening iron;
(g) apply to the hair a fixing composition comprising an oxidizing agent for performing an oxidative post-treatment of the hair; and
(h) rinse the hair with water.

### Results

The permanent shaping is assessed by three hair stylists, using an assessment scale from 1 (poor) to 5 (excellent). The assessment below is the average of the scores obtained.

| Criteria | C1 (reference) | C3 |
|---|---|---|
| Mass appearance and odour | 1.67 | 4.33 |
| Application | 3.33 | 4.33 |
| Comb through | 1.67 | 4.00 |
| Rinsability | 1.67 | 4.33 |

Examples C4 and C5 describe compositions with ratio fatty alcohols/non-ionic surfactants of 10:0.9. This ratio of fatty alcohols/non-ionic surfactants and all ratios higher than this do not provide a good gel network as early signs of phase separation appear evident few hours/days after the composition production.

Variations and modifications of the invention and further embodiments thereof, in addition to those described herein, will become apparent to those skilled in the art from the full contents of this document. The subject matter herein contains information, exemplification and guidance that can be adapted to the practice of this invention in its various embodiments and equivalents thereof. It is intended that the appended claims cover all such variations, modifications, embodiments and equivalents.

1. A permanent shaping composition for the permanent deformation of hair comprising, in a cosmetically acceptable carrier:
(a) at least one reducing agent comprising a mercapto functional group;
(b) at least one alkalizing agent;
(c) at least one fatty alcohol;
(d) at least one non-ionic surfactant different from the fatty alcohol;
wherein the at least one fatty alcohol and the at least one non-ionic surfactant are present in a weight ratio from about 10:3.5 to 10:1, preferably from about 10:3 to about 10:1.5, more preferably of about 10:2.5 to 10:2.0.

2. A composition, according to claim 1, wherein the cosmetically acceptable carrier is an aqueous carrier, preferably an aqueous carrier selected from the group consisting of water, water-soluble polyhydric alcohols and mixtures thereof.

3. A composition, according to claim 1, wherein the reducing agent is selected from the group consisting of thioglycolic acid, thiolactic acid and their salts thereof, such as their ammonium and ethanolamine salts; cysteine and its salts thereof, such as hydrochloride cysteine; homocysteine; cysteamine and its salts thereof; N-acetyl cysteine; thioglycerol; ethanediol monothioglycolate; 1,2-propyleneglycol monothioglycolate; 1,3-propanediol monothioglycolate or the isomer mixture resulting therefrom; 1,3-butanediol, 1,4-butanediol monothioglycolate and their isomer mixtures therefrom; polyethylene glycol monothioglycolates, such as di-, tri- and tetraethyleneglycol monothioglycolates; glycerol monothiolactate; mercapto carboxylic acids and esters thereof; N-alkyl-2-mercaptoacetamides; and mixtures thereof; preferably from the group consisting of cysteamine, cysteine, thioglycolic acid, their salts thereof and mixtures thereof.

4. A composition, according to claim 1, wherein it comprises a total amount of reducing agent ranging from about 0.1 to about 35%, preferably from about 0.5 to about 20%, more preferably about 1 to about 15%, of a reducing agent, by total weight of the composition.

5. A composition, according to claim 1, wherein the alkalizing agent is selected from the group consisting of ammonia, ammonium carbamate, ammonium(bi)carbonate, monoethanolamine, diethanolamine, triethanolamine, 2-amino-2-methyl-1-propanol, 2-amino-2-methyl-1,3-propanediol, 2-amino-2-ethyl-1,3-propanediol, 2-amino-1-butanole, tris(hydroxylmethyl)-aminomethane, tris-(2-hydroxypropyl)-amine, lysine, 2,2-iminobisethanol, iminourea (guanidine carbonate), tetrahydro-1,4-oxazine, 2-amino-5-guanidin valeric acid, 2-aminoethansulfonic acid, N-methyl ethanolamine, isopropanolamine, diisopropanolamine, triisopropanolamine, glucamine, sodium hydroxide, potassium hydroxide, lithium hydroxide, magnesium hydroxide, other water-soluble physiologically tolerable salts of organic and inorganic bases, and mixtures thereof; preferably the alkalizing agent is selected from the group consisting of ammonia, monoethanolamine, and mixture thereof.

6. A composition, according to claim 1, wherein it comprises a total amount of alkalizing agent ranging from about 0.1 to about 10%, preferably from about 0.2 to about 5%, more preferably from 0.25 to 4% by total weight of the composition.

7. A composition, according to claim 1, wherein the fatty alcohol is selected from the group consisting of linear and/or branched C₁₄ to C₃₀ fatty alcohols; preferably from the group consisting of linear C₁₄ to C₃₀ fatty alcohols; more preferably from the group consisting of cetyl alcohol, stearyl alcohol, cetearyl alcohol, behenyl alcohol, and mixtures thereof; still more preferably the fatty alcohol is cetearyl alcohol.

8. A composition, according to claim 1, wherein it comprises a total amount of fatty alcohol ranging from about 0.5 to about 20%, preferably from about 2 to about 15%, more preferably from about 4 to about 12%, still more preferably from about 5 to about 10% by total weight of the composition;

9. A composition, according to claim 1, wherein the non-ionic surfactant is selected from the group consisting of polyoxyethylene C₈ to C₃₀ alkyl ethers; preferably from the group consisting of polyoxyethylene C₈ to C₃₀ alkyl ethers having at least 2 ethylene oxide units, or at least 5 ethylene oxide units; more preferably from the group consisting of polyoxyethylene C₈ to C₃₀ alkyl ethers having from 10 to 50 ethylene oxide units; still more preferably from the group consisting of polyoxyethylene C₈ to C₃₀ alkyl ethers having from 15 to 30 ethylene oxide units; still more preferably from the group consisting of ceteareth-25, steareth-20, and mixtures thereof; most preferably the non-ionic surfactant is steareth-20.

10. A composition, according to claim 1, wherein it comprises a total amount of non-ionic surfactant other than fatty alcohol ranging from about 0.2 to about 5%, preferably from about 0.5 to about 4%, more preferably from about 1.1 to about 2.2%, by total weight of the composition.

11. A composition, according to claim 1, wherein it comprises a total amount of fatty alcohol and non-ionic surfactant ranging from about 0.7 to about 25%, preferably from about 2.5 to about 19%, more preferably from about 6.1 to about 12.2%, by total weight of the composition; and wherein the fatty alcohol and the non-ionic surfactant are comprised, in part or all, in a gel network system.

12. A composition, according to claim 1, wherein it is free of anionic surfactant and/or cationic surfactant.

13. A composition, according to claim 1, wherein it comprises a thickener and/or rheology modifier, a liquid oil, a solid fat, a cationic polymer, a silicone, a swelling agent, a disulfide, and mixtures thereof.

14. A composition, according to claim 1, wherein it comprises an additive selected from the group consisting of alcohols, sugar alcohols, preservatives, antioxidants, sequestering agents, salts, pearlescent aids, UV-filters and sunscreens, pH adjusting agents, opacifiers, solubilizers, perfume oils, dyes, aminoacids, hydrolysed proteins, and mixtures thereof.

15. A kit for the permanent deformation of hair comprising:
(a) an individually packaged permanent shaping composition according to claim 1;
(b) an individually packaged fixing composition comprising an oxidizing agent.

16. A method for the permanent deformation of hair comprising the following steps:
(a) providing a permanent shaping composition according to claim 1;
(b) applying the permanent shaping composition to the hair before or after putting the hair in a desired shape;
(c) allowing the permanent shaping composition to act on the hair for a predetermined acting time sufficient for the permanent shaping of the hair;
(d) rinsing the hair with water;
(e) applying to the hair a fixing composition comprising an oxidizing agent for performing an oxidative post-treatment of the hair; and
(f) rinsing the hair with water.
